(19) United States
(12) Patent Application Publication
(10) Pub. No.: US 2009/0078272 A1
(43) Pub. Date: Mar. 26, 2009

(54) Making elastic body-contact articles with studded surfaces to be used as indicia carriers

(76) Inventor: Alexander Crispino, Staten Island, NY (US)

Correspondence Address: Aleksandr Smushkovich, POB 140505, Brooklyn, NY, 11214, US

(21) Appl. No.: 11/903,909

(22) Filed: Sep. 25, 2007

(51) Int. Cl. A61F 6/04

(52) U.S. Cl. 128/844

(57) ABSTRACT

Indicia carriers for display of advertisements or brand/trade marks are provided comprising an elastic base, e.g. condoms, gloves, etc., and a plurality of indicia studs coupled to the base, and arranged in lines, contours, or patterns, forming images of symbols, characters, etc. to display the advertisements or brand/trade marks, wherein the images made sufficient for visual resolution of an average person. Alternatively, the carrier further includes a plurality of background studs, coupled to the base, surrounding the indicia studs, and having a size or color different from a size or color of the indicia studs, so that the difference in the sizes/colors is sufficient to provide easy visual resolution of the images for an average person. Optionally, the studs can be made of or covered by materials glowing in the dark.

### TECHNICAL FIELD

The present invention relates to indicia carriers, particularly to the making and using elastic studded surfaces of latex or thin-rubber articles as indicia carriers, such as, for example, advertising and brand/trade mark carriers.

### BACKGROUND OF THE INVENTION

A multitude of indicia carriers are known in the prior art, including various articles, and parts thereof. For example, advertisements and brand/trade marks are placed on commercial signs, posters, trucks, industrial machines, buildings' walls, clothing (e.g. T-shirts, jackets, etc.), bags, packaging, cardboards, paper (not only newspapers and books, but also cash-register ribbons, wall-paper, etc.), paper tissue (e.g. paper towels, toilet tissue), and so on. Even fog emitting materials are now used for ad writing in the sky.

On the other hand, there are known latex and thin-rubber articles having elastic studs on their surfaces. For example, an Internet website http://www.2beaut.com/others.html describes ribbed or countered dotted condoms, whose surfaces are covered by specific elastic ribs or elastic raised studs for increasing sensitivity.

U.S. Pat. No. 4,852,586 teaches “ . . . a plurality of pairs of shaped projections spaced apart and arranged to form, juxtaposed or apposing pairs, integrally formed with, and coaxially extending from, the opposite sides of a base, prophylactically impermeable, flexible sheet material or pellicle. In a preferred embodiment, the projection pairs are generally cylindrical in shape. It will be appreciated that in the preferred embodiment, and particularly for manufacture of the condoms and gloves of the invention, the prophylactically impermeable sheet material is of thin, latex rubber or other inert elastomeric material.”

The present invention is dedicated to further widening the variety of types of articles' surfaces that may be utilized as indicia carriers, particularly as advertisement carriers or brand/trade mark carriers.

### SUMMARY OF THE INVENTION

This invention proposes specifically designed studded latex or thin-rubber articles utilized with immediate contact to a human body, herein further called ‘body-contact articles’, such as condoms, gloves, stockings, etc. to be used as indicia carriers. Several exemplary embodiments of the inventive body-contact articles are disclosed in the present description.

The invention provides indicia carriers for display of advertisements or brand/trade marks comprising an elastic base surface of a body-contact article, and a plurality of indicia studs coupled to the base surface, and arranged in lines, contours, or patterns, forming images of symbols, characters, etc. to display the advertisements or brand/trade marks, wherein the images made sufficient for visual resolution of an average person. Alternatively, the carrier further includes a plurality of background studs, coupled to the base, surrounding the indicia studs, and having a size or color different from a size or color of the indicia studs, so that the difference in the sizes/colors is sufficient to provide easy visual resolution of the images for an average person. Optionally, the studs can be made of or covered by materials glowing in the dark.

### BRIEF DESCRIPTION OF DRAWINGS

FIG. 1 illustrates an isometric view of a piece of body-contact article, made according to a first preferred embodiment of the invention.

FIG. 2 illustrates an isometric view of a piece of body-contact article, made according to a second preferred embodiment of the invention.

FIG. 3 illustrates an isometric view of a piece of body-contact article, made according to a third preferred embodiment of the invention.

Identical reference numerals on the drawings generally refer to the same elements on different figures. A first-time introduced numeral in the description is enclosed into parentheses.

### DESCRIPTION OF SAMPLE PREFERRED EMBODIMENTS OF THE INVENTION

While the invention may be susceptible to embodiment in different forms, there are shown in the drawings, and will be described in detail herein, specific embodiments of the present invention, with the understanding that the present disclosure is to be considered an exemplification of the principles of the invention, and is not intended to limit the invention to that as illustrated and described herein.

### First Embodiment

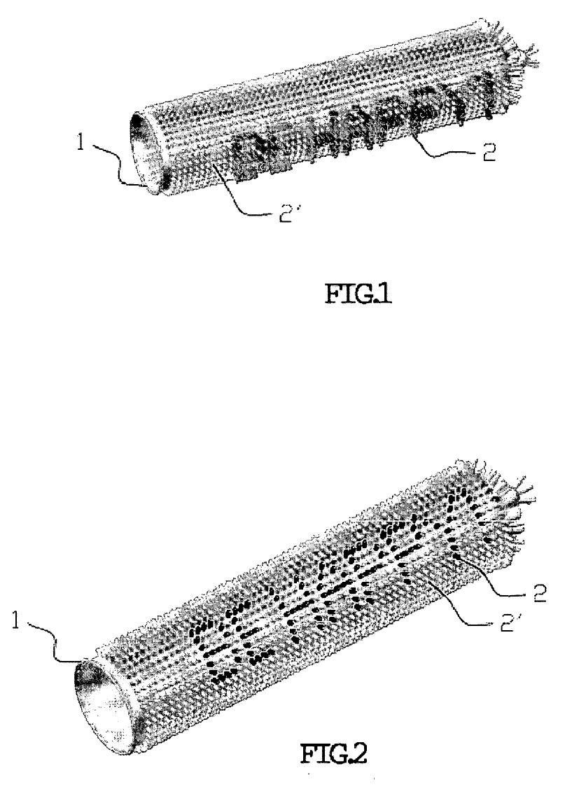

A first preferred embodiment of a body-contact article according to the present invention is illustrated on FIG. 1. The inventive body-contact article comprises a base surface (1), generally made of elastic material, such as latex or thin-rubber (including synthetic rubber), and a plurality of indicia studs (2), coupled to the base surface. The indicia studs are made with a first predetermined size, and preferably made of the same material as the base surface 1. In this context, the ‘predetermined size’ is defined as either one: a predetermined height of the stud, a predetermined width (diameter) of the stud, both a predetermined height and predetermined width (diameter) of the stud.

The inventive body-contact article generally comprises a plurality of background studs (2′) preferably made of the same material with a second predetermined size. The indicia studs 2 and background studs 2′ each is preferably shaped as a cylinder with a semi-spherical head, and can be produced by means of known technologies. Other shapes of the studs can be utilized if needed.

The first embodiment of the invention is characterized in that the first predetermined size of the indicia studs 2 is chosen greater than the second predetermined size of the background studs 2′. FIG. 1 shows the studs 2 with the height greater than the height of the studs 2′. In other embodiments, the size relationship can be opposite.

The indicia studs 2 are arranged in lines, contours, or patterns, so that forming images of symbols, characters, etc. necessary to display the advertisement or brand/trade mark. The background studs 2′ substantially surround the indicia studs 2. The difference in th predetermined sizes of the studs 2 and 2′ should generally be chosen sufficient to provide easy visual resolution of the images for an average person.

### Second Embodiment

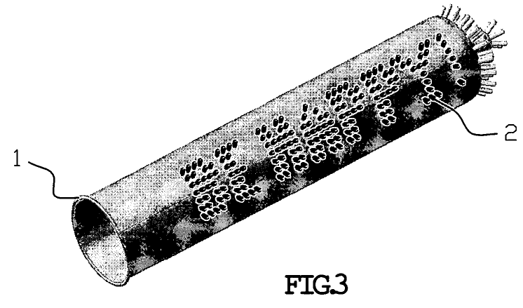

A second preferred embodiment of a body-contact article according to the present invention is illustrated on FIG. 2. The inventive body-contact article comprises a base surface 1, and a plurality of indicia studs 2 preferably made of the same material with a first predetermined color.

The inventive body-contact article generally comprises a plurality of background studs 2′ preferably made of the same material with a second predetermined color. The background studs 2′ substantially surround the indicia studs 2. The indicia studs 2 and background studs 2′ are coupled to the base surface 1, and each is preferably shaped as a cylinder with a semi-spherical head, similar to the studs of the first embodiment.

The second embodiment of the invention is characterized in that the first predetermined color of the indicia studs 2 is chosen contrasted with the second predetermined color of the background studs 2′.

The indicia studs 2 are arranged in lines, contours, or patterns, so that forming images of symbols, characters, etc. necessary to display the advertisement or brand/trade mark. The contrast of the predetermined colors of the studs 2 and 2′ should generally be chosen sufficient to provide easy visual resolution of the images for an average person.

### Third Embodiment

A third preferred embodiment of a body-contact article according to the present invention is illustrated on FIG. 3. The inventive body-contact article comprises a base surface 1, and a plurality of indicia studs 2 preferably made of the same material, and coupled to the base surface 1.

The third embodiment of the invention is characterized in that there are no background studs. The indicia studs 2 are arranged in lines, contours, or patterns, so that forming images of symbols, characters, etc. necessary to display the advertisement or brand/trade mark. The images should generally be so chosen to provide sufficient visual resolution for an average person.

### Optional Embodiments

In some other embodiments (not illustrated), either the indicia or the background studs can be made of or covered by materials glowing in the dark. Alternatively, the indicia and background studs can have different cross-section shapes, e.g. a circle and a hexagon.

It should be understood that each of the elements described above, or two or more together, may also find a useful application in other types of constructions differing from the types described above.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

I claim:

1. An indicia carrier for display of advertisements or brand/trade marks comprising
an elastic base surface, and
a plurality of indicia studs coupled to said base surface, and arranged in lines, contours, or patterns, so that forming images of symbols, characters, etc. necessary to display the advertisement or brand/trade mark, wherein the images made sufficient to provide visual resolution for an average person.

2. The indicia carrier, according to claim 1, wherein said base surface and said indicia studs made of the same material.

3. The indicia carrier, according to claim 1, wherein
said indicia studs made with a first predetermined size;
said carrier further comprising
a plurality of background studs made with a second predetermined size coupled to said base surface, the background studs substantially surrounding the indicia studs; and
wherein the first predetermined size of said indicia studs chosen different from the second predetermined size of said background studs, so that the difference in the first and second predetermined sizes sufficient to provide easy visual resolution of the images for an average person.

4. The indicia carrier, according to claim 3, wherein said indicia studs and said background studs each shaped as a cylinder with a semi-spherical head.

5. The indicia carrier, according to claim 3, wherein said indicia studs and said background studs having different cross-section shapes.

6. The indicia carrier, according to claim 1, wherein
said indicia studs made with a first predetermined color;
said carrier further comprising
a plurality of background studs made with a second predetermined color, coupled to said base surface, the background studs substantially surrounding the indicia studs; and
wherein the first predetermined color of said indicia studs chosen contrasted to the second predetermined color of said background studs, so that the contrast being sufficient to provide easy visual resolution of the images for an average person.

7. The indicia carrier, according to claim 6, wherein said indicia studs and said background studs each shaped as a cylinder with a semi-spherical head.

8. The indicia carrier, according to claim 6, wherein said indicia studs and said background studs having different cross-section shapes.

9. The indicia carrier, according to claim 6, wherein either the indicia studs or the background studs made of or covered by materials glowing in the dark.

10. The indicia carrier, according to claim 3, wherein either the indicia studs or the background studs made of or covered by materials glowing in the dark.
